(19) United States
(12) Patent Application Publication
(10) Pub. No.: US 2020/0000393 A1
(43) Pub. Date: Jan. 2, 2020

(54) METHOD AND DEVICE FOR MONITORING BODY FLUID DISCHARGE

(71) Applicants: YI-CHIH CHENG, Kaohsiung (TW); CHIH-HSIANG CHANG, Hsinchu (TW); ZI-YANG LIN, Taoyuan (TW); NING CHEN, Taoyuan (TW); CHIEN-HSING LIN, Kaohsiung (TW); TIEN-YU HSIN, Tainan (TW)

(72) Inventors: YI-CHIH CHENG, Kaohsiung (TW); CHIH-HSIANG CHANG, Hsinchu (TW); ZI-YANG LIN, Taoyuan (TW); NING CHEN, Taoyuan (TW); CHIEN-HSING LIN, Kaohsiung (TW); TIEN-YU HSIN, Tainan (TW)

(21) Appl. No.: 16/419,621

(22) Filed: May 22, 2019

(30) Foreign Application Priority Data
Jun. 29, 2018 (TW) 107122695

(51) Int. Cl. A61B 5/20

(52) U.S. Cl. CPC A61B 5/208; A61B 2562/16

(57) ABSTRACT

A device for monitoring body fluid discharge almost in real time is provided, the device includes a base, a motor, a catheter holder, and a liquid level sensor. The motor is fixed on the base and includes an output shaft. The catheter holder is connected to the output shaft and is rotatable relative to the base by the output shaft. The catheter holder includes a curved holding chamber with two openings containing a catheter. The liquid level sensor is disposed at one of the openings for detecting a liquid level of body fluid in the catheter and such level is recorded when a fully-filled catheter is automatically tipped and emptied into a collecting bag. A method for monitoring body fluid discharge using the device is also provided.

### FIELD

The subject matter herein generally relates to medicine and medical equipment.

### BACKGROUND

In Kidney Diseases such as Acute Kidney Injury (AKI), the discharge volume of urine is measured. However, there is no known device that can perform real-time urine measurement. Medical guidelines suggest nurses pour urine out of a urine bag when taking a shift. Such a method of urine output monitoring is inaccurate, time-consuming, and wasteful of human resource.

Thus, there is a need for a device and a method for facilitating monitoring of body fluid discharge with an improved accuracy.

### SUMMARY

A device for monitoring body fluid discharge is provided, the device includes a base, a motor, a catheter holder, and a liquid level sensor. The motor is fixed on the base and includes an output shaft. The catheter holder is connected to the output shaft and rotatable relative to the base by the output shaft. The catheter holder includes a curved holding chamber with two openings for receiving a catheter. The liquid level sensor is disposed at one of the openings for detecting a liquid level of body fluid in the catheter.

A method for monitoring body fluid discharge using the device is also provided. The method mounts a catheter in the catheter chamber and forms a buffer zone in the catheter, senses whether the body fluid in the buffer zone reaches a predefined maximum position, and rotates the catheter holder relative to the base to pour the body fluid in the fully-filled buffer zone to a body fluid collection bag.

The device and method for monitoring body fluid discharge of the present disclosure employ a catheter holder to form a buffer zone on a catheter to store body fluid. When a liquid level sensor detects that the body fluid in the buffer zone reaches a predefined maximum position, the liquid level sensor triggers a motor to rotate the catheter holder to pour the body fluid to a body fluid collection bag connected to an end of the catheter, thereby facilitating the monitoring of body fluid discharge.

### BRIEF DESCRIPTION OF THE DRAWINGS

Implementations of the present technology will now be described, by way of embodiments, with reference to the attached figures.

FIG. 1 is an isometric view of a device for monitoring body fluid discharge according to a first embodiment of the present disclosure.

FIG. 2 is an exploded view of the device of FIG. 1.

FIGS. 3 and 4 are front views of the device of FIG. 1 in use.

FIGS. 5 and 6 are side views showing a rotation of the device of FIG. 1.

FIG. 7 is an isometric view of a device for monitoring body fluid discharge according to a second embodiment of the present disclosure.

FIG. 8 is an isometric view of a device for monitoring body fluid discharge according to a second embodiment of the present disclosure, with cover opened.

FIG. 9 is a front side view of the device of FIG. 8.

FIG. 10 is a front side view of the device of FIG. 8, with closed cover (shown as dotted line).

FIG. 11 is a front side view of the device of FIG. 8, with a catheter holder being rotated to pour body fluid.

FIG. 12 is an isometric view of a device for monitoring body fluid discharge according to a third embodiment of the present disclosure.

FIG. 13 is an isometric view of the device of FIG. 12, with cover opened.

FIG. 14 is an isometric view of the device of FIG. 12, with several components removed.

FIGS. 15 and 16 are exploded views of the device of FIG. 12 from a different view point.

FIG. 17 is an isometric view of a device for monitoring body fluid discharge according to a forth embodiment of the present disclosure.

FIG. 18 is an isometric view of the device of FIG. 17, with cover opened.

FIGS. 19 and 20 are exploded views of the device of FIG. 17 from different view points, with cover removed.

FIG. 21 is an isometric view showing an engagement of a movable piece and a first seat part of the device of FIG. 17.

FIG. 22 is an exploded view of part elements of the device of FIG. 17

FIG. 23 is a flowchart of a method for monitoring body fluid discharge according to an embodiment.

FIG. 24 is a diagram showing a relationship of a volume of body fluid discharged to a buffer zone of a catheter and time.

### DETAILED DESCRIPTION

The present disclosure is made in conjunction with the accompanying drawings. Specific embodiments of the present disclosure are described.

In the following description, when an element is described as being “fixed to” another element, the element can be fixed to the another element with or without intermediate elements. When an element is described as “connecting” another element, the element can be connected to the another element with or without intermediate elements.

Without given definition, all terms given below are of the same meaning as commonly understood by those skilled in the art. The term “and/or” means including any and all combinations of one or more of associated listed items.

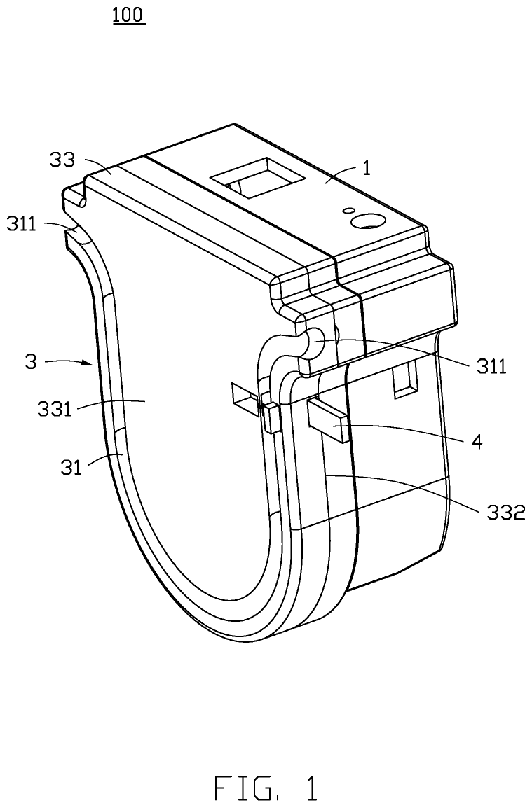

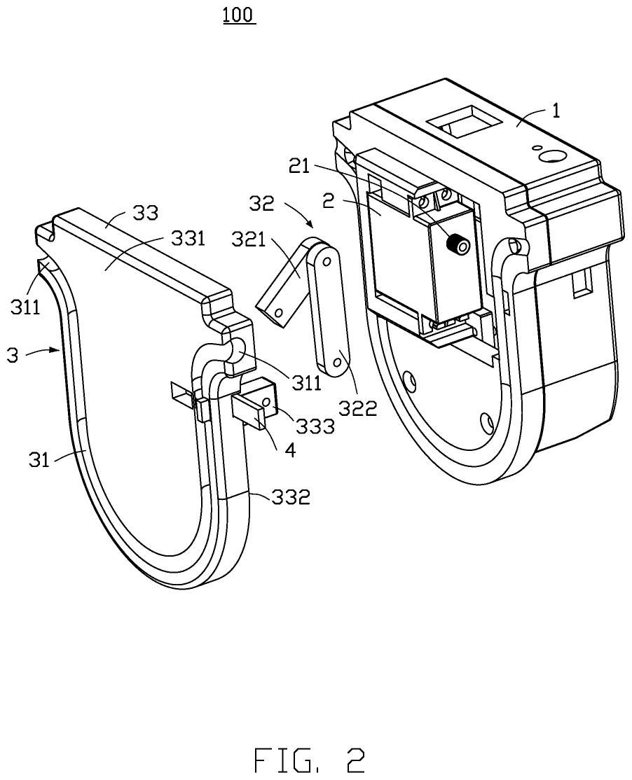

Referring to FIGS. 1 and 2, a device for monitor body fluid discharge 100 (hereinafter “the device 100”) includes a base 1, a motor 2, a catheter holder 3, and a liquid level sensor 4.

The base 1 can be disposed directly or by a fixing frame (not shown) anywhere that facilitates the monitoring of body fluid discharge. In one embodiment, the base 1 can be attached to a bed of a patient, or be attached to the bottom of toilet, the bottom of a mobile urine collecting device such as a diaper or a chamber pot, or the opening of a urine container such as a plastic or paper cup, or a test tube.

The color of the device 100 can be colored to match colors of wards and beds.

The motor 2 is fixed on the base 1 and includes an output shaft 21.

The catheter holder 3 is connected to the output shaft 21 and driven by the output shaft 21 to rotate relative to the base 1. The catheter holder 3 includes a curved holding chamber 31. The holding chamber 31 has two openings 311. The holding chamber 31 can be any curved shape, such as U-shape.

The liquid level sensor 4 is disposed at one of the opening 311. The liquid level sensor 4 can be a light sensor for detecting a liquid level.

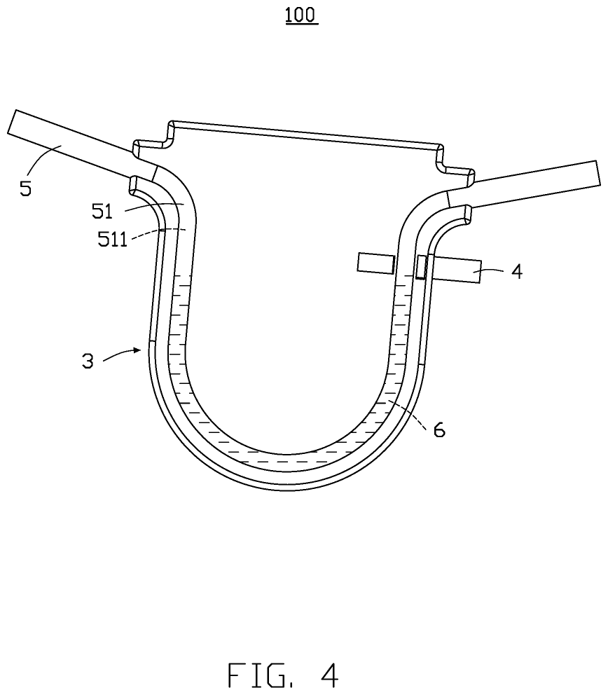

Referring to FIGS. 1-3, when in use, a catheter 5 is held in the holding chamber 31 such that the catheter 5 forms a buffer zone 511 for storing body fluid. The ends of the catheter 5 extend outside of the holding chamber 31 through the openings 311. Referring to FIG. 4, when the body fluid 6 in the buffer zone 511 reaches a predefined maximum position which can be detected by the liquid level sensor 4, the liquid level sensor 4 sends a signal to trigger the motor 2 to rotate. The motor 2 rotates the catheter holder 3 relative to the base 1, such that the body fluid 6 in the catheter 3 flow into a body fluid collection bag (not shown) connected to an end of the catheter 3. After the body fluid 6 is emptied out to the body fluid collection bag, the motor 2 rotates the catheter holder 3 back to the original position and the catheter 5 can collect body fluid 6 again. The device 100 further includes a timing unit (not shown). The timing unit is also triggered by the signal sent by the liquid level sensor 4, to record a current time point as a discharge time. The timing unit keeps record a discharge time each time the body fluid 6 in the buffer zone 511 reaches a predefined maximum position, thus to facilitate the monitoring of body fluid (such as blood or urine) discharge.

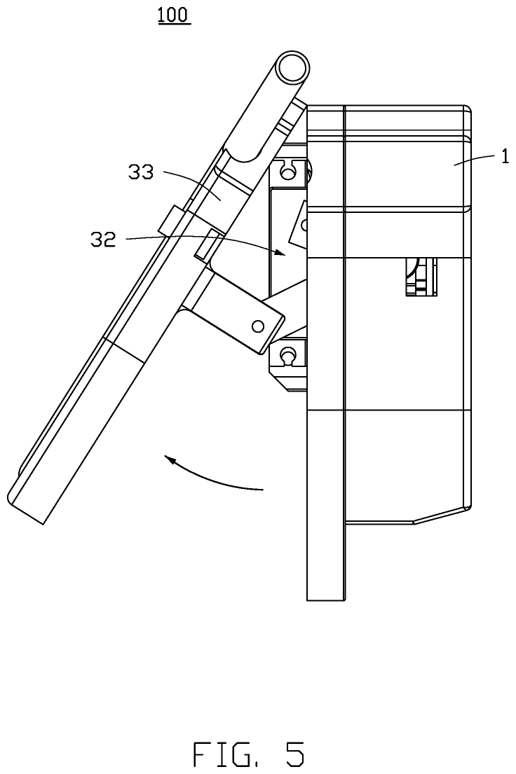

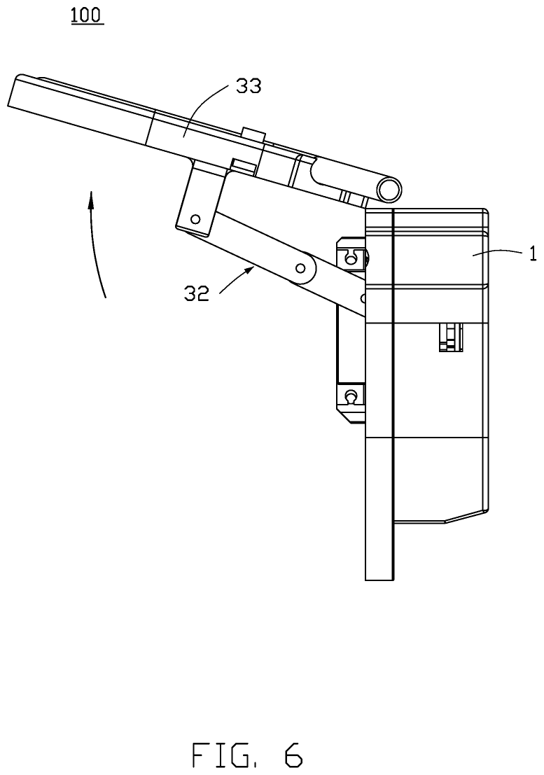

FIGS. 1-6 relate to a first embodiment. Referring to FIG. 2, the catheter holder 3 further includes a linkage assembly 32 and a pivot seat 33. The linkage assembly 32 pushes or pulls the pivot seat 33 to rotate relative to the base 1. The pivot seat 33 has an appropriate weight or structure which allows the pivot seat 33 to be rotated relative to the base 1 by the linkage assembly 32. In the present embodiment, an upper edge of the pivot seat 33 is pivotally connected to the base 1 and a bottom portion of the pivot seat 33 can be rotated relative to the base 1 by the linkage assembly 32. The linkage assembly 32 includes a first linkage rod 321 and a second linkage rod 322. The first linkage rod 321 has one end connected to the output shaft 21 of the motor 2, and another end connected to an end of the second linkage rod 322. The pivot seat 33 includes a front side 331 and a back side 332 opposite to the front side 331. The holding chamber 31 is formed at the front side 331 and a connecting piece 333 is formed at the back side 332. The connecting piece 333 is connected to another end of the second linkage rod 322.

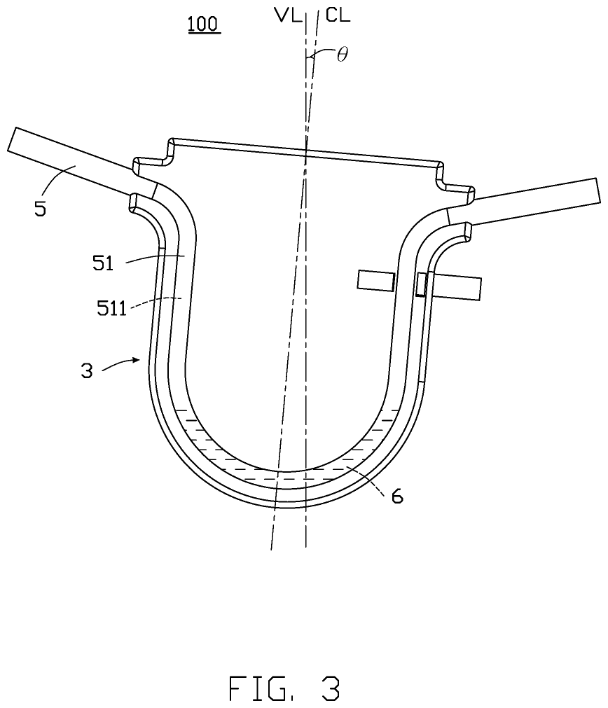

Referring to FIG. 3, when in use, the device 100 is tilted, and an angle is formed between a central line CL of the device 100 and a vertical line VL. The angle is about 4-20 degrees from vertical. The tilting of the device 100 facilitates the flowing of the body fluid 6 to the body fluid collection bag. Referring to FIGS. 4-6, when the liquid level sensor 4 detects that the body fluid 6 in the buffer zone 511 reaches the predefined maximum position, the liquid level sensor 4 triggers the motor 2 to rotate, the linkage assembly 32 is driven by the motor 2 to rotate the pivot seat 33 relative to the base 1, which results in the flowing of the body fluid 6 to the body fluid collection bag.

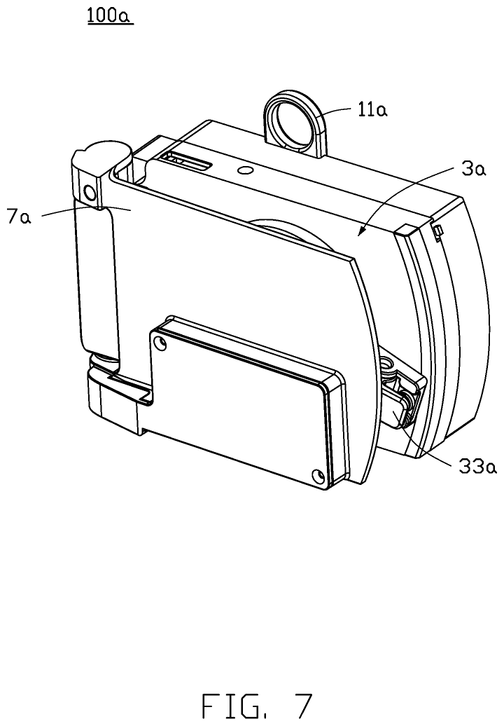

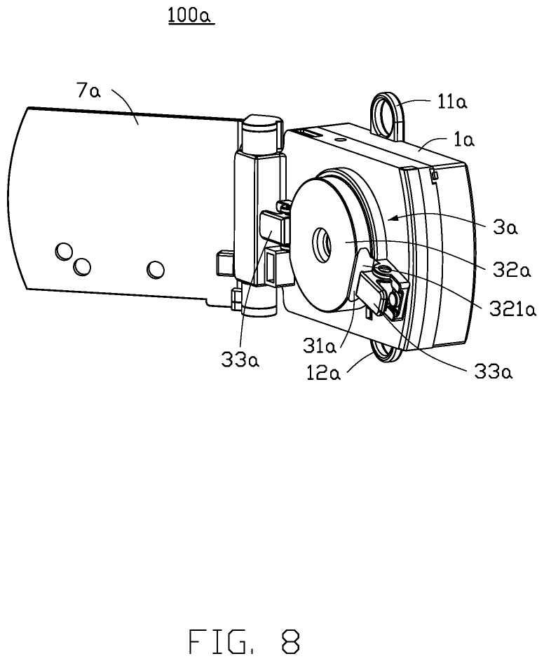

FIGS. 7-11 relate to a second embodiment. Referring to FIGS. 7 and 8, a device for monitoring body fluid discharge 100a (hereinafter “the device 100a”) includes a base 1a and a catheter holder 3a. The base la is formed with an upper hanging ring 11a and a lower hanging ring 12a. The catheter holder 3a is a rotatable disk located on the base 1a. The catheter holder 3a includes a disk 32a and a plurality of spring clips 33a mounted around the disk 32a. A circumferential surface of the disk 32a facing the spring clips 33a defines grooves 321a, each groove 321a corresponds to one of the spring clips 33a. A holding chamber 31a for holding a catheter 5a is formed between the disk 32a and the spring clips 33a. In the present embodiment, there are two spring clips 33a mounted around the disk 32a and symmetrical about the disk 32a.

The device 100a further includes a cover 7a pivotally connected to the base la.

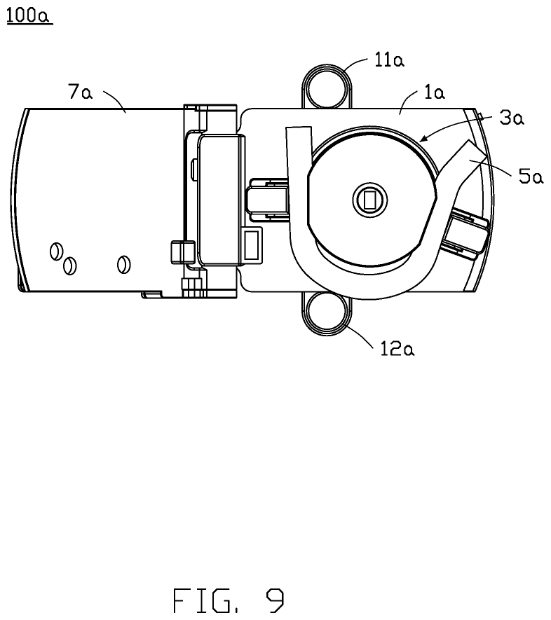

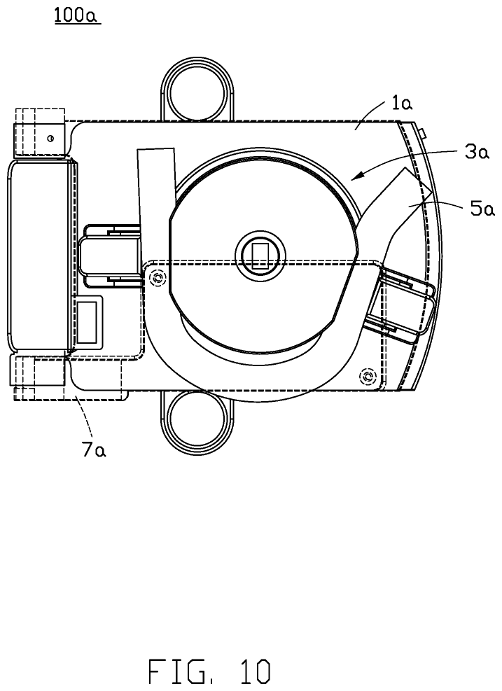

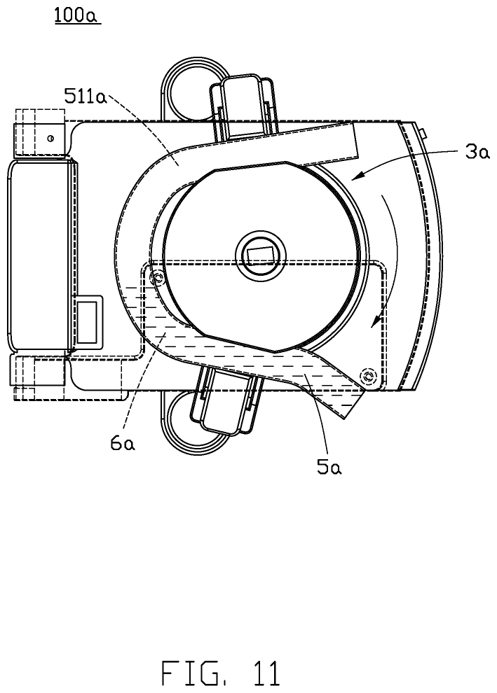

Referring to FIG. 9, when in use, the upper hanging ring 11a is hung on a drip stand (not shown), and a body fluid collection bag is hung on the lower hanging ring 12a. The cover 7a is opened by rotating relative to the base 1a, such that the catheter 5a is able to be mounted in the holding chamber 31a. Referring to FIG. 10, the cover 7a is closed, and the cover 7a closes over the catheter 5a to seal the catheter 5a against spillage during use. Referring to FIG. 11, when body fluid 6a in a buffer zone 511a of the catheter 5a reaches a predefined maximum position, the catheter holder 3a is rotated to flow the body fluid 6a in the catheter 5a to the body fluid collection bag connected to an end of the catheter 5a.

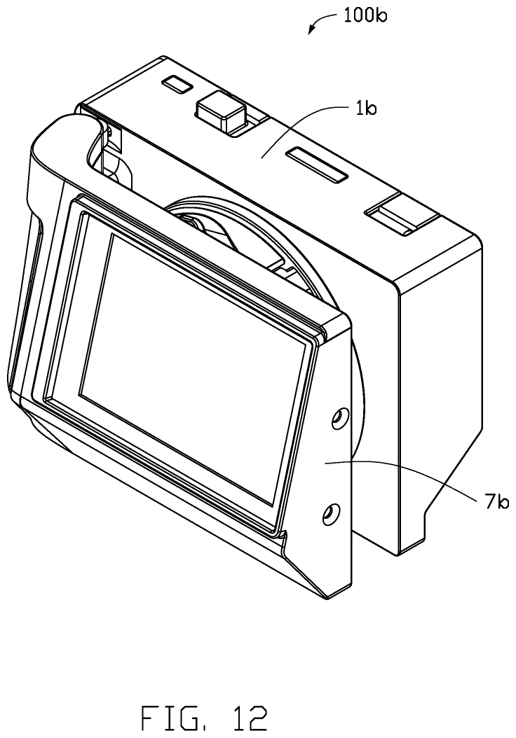

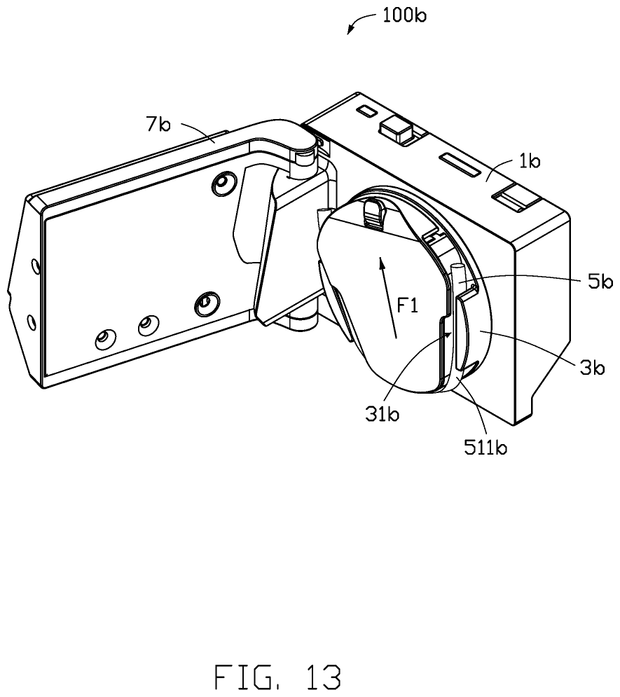

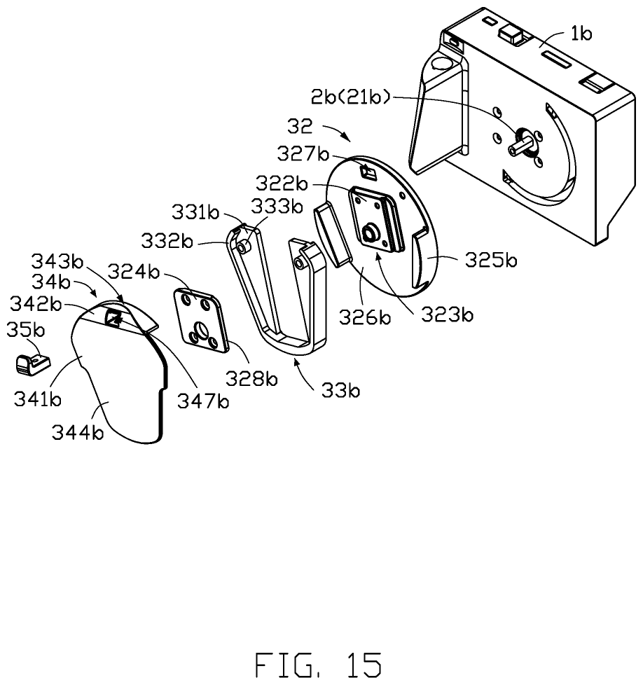

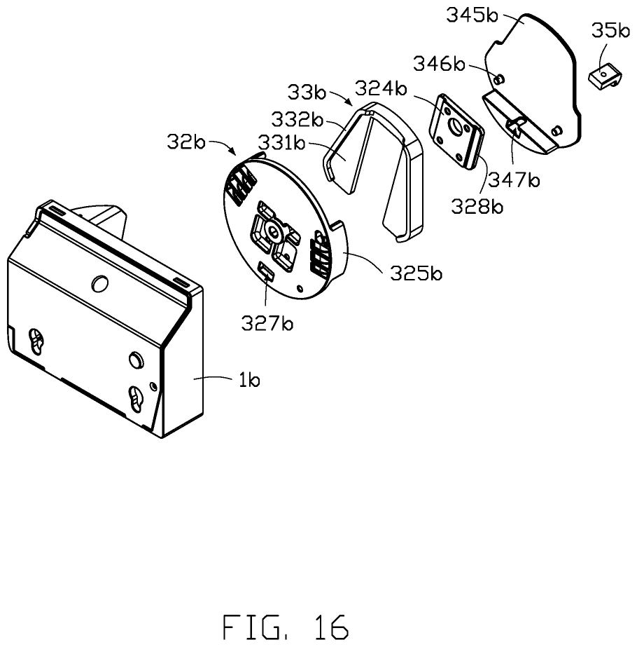

FIGS. 12-16 relate to a third embodiment. Referring to FIGS. 12 and 13, a device for monitoring body fluid discharge 100b (hereinafter “the device 100b”) includes a base 1b, a motor 2b, a catheter holder 3b, and a cover 7b. The motor 2b is fixed on the base 1b and includes an output shaft 21b. The catheter holder 3b is a rotatable disk 32b located on the base 1b and rotatable by the output shaft 21b of the motor 2b. The cover 7b is pivotally connected to the base 1b and can be rotated to open to expose the catheter holder 3b or to cover over the catheter holder 3b. When the cover 7b is opened, a catheter 5b can be mounted to a holding chamber 31b of the catheter holder 3b. When the cover 7b is closed, the cover 7b closes over the catheter 5b to seal the catheter 5a against spillage during use.

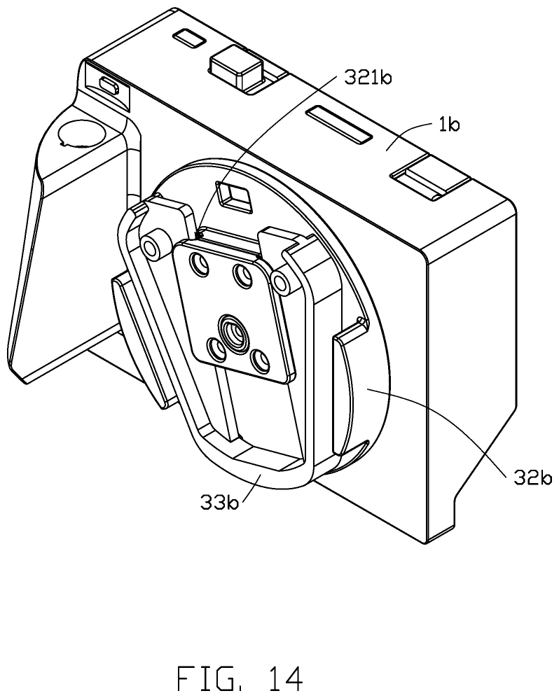

Referring to FIGS. 14-16, the catheter holder 3b includes a seat 32b, a movable piece 33b movable on the seat 32b, and a holding cover 34b. The seat 32b is located on the base 1b and is rotatable by the output shaft 21b. The seat 32b includes at least one guiding groove 321b along which the movable piece 33b can move forwards and backwards. In the embodiment, the seat 32b includes two guiding grooves 321b. The guiding grooves 321b are formed by a platform 322b protruding from a central portion 323b of the seat 32b and a pressing plate 324b covering the platform 322b. The pressing plate 324b has a size larger than that of the platform 322b, two opposite edges 328b exceed beyond the platform 322b so that the guiding grooves 321b are formed at opposite sides of the platform 322b. The pressing plate 324b is mounted on the platform 322b by screws or other. The pressing plate 324b can be integrated with the platform 322b by molding technology in another embodiment. The seat 32b further includes at least one protruded block 325b (two protruded blocks 325b are shown in this embodiment) located at a surrounding portion 326b of the seat 32b. The protruded blocks 325b are symmetrically distributed on opposite sides of the platform 322b and the pressing plate 323b, and are apart from the guiding grooves 321b.

The movable piece 33b includes at least one guiding block 331b receivable and movable in the at least one guiding groove 321b of the seat 32b, such that the movable piece 33b can be moved by users along the guiding groove 321b forwards and backwards. In the present embodiment, the movable piece 33b includes two opposite guiding blocks 331b each corresponding to a guiding groove 321b. When the movable piece 33b engages with the seat 32b by receiving the guiding blocks 331b in the guiding grooves 321b, the movable piece 33b is located between the protruded blocks 325b. A curved holding chamber 31b for holding a catheter 5b is formed between the movable piece 33b and the protruded blocks 325b. The movable piece 33b can be moved forward following the direction of arrow F1 to increase a volume of the holding chamber 31b, so that the catheter 5b can be placed in the holding chamber 31b more easily. The movable piece 33b can be moved backward to decrease the volume of the holding chamber 31b, so that the catheter 5b can be tightly held in the holding chamber 31b. In the present embodiment, the movable piece 33b is U-shaped and has two legs 332b. The guiding blocks 331b are formed on the legs 332b and extend towards each other.

The holding cover 34b is fixed on the movable piece 33b and covers the movable piece 33b. The holding cover 34b includes two flanks 341b located on opposite sides of the holding cover 34b. The flanks 341b cover the catheter 5b when the catheter 5b is received in the holding chamber 31b and prevent the catheter 5b from sliding out of the holding chamber 31b. The flanks 341b are symmetrical in this embodiment but may be asymmetrical in another embodiment. The holding cover 34b further includes a curved surface 342b located at a front end 343b of the holding cover 34b. The curved surface 342b facilitates the application of a downward force on the holding cover 34b when operating the holding cover 34b. The holding cover 34b includes a front side 344b away from the movable piece 33b and a back side 345b facing the movable piece 33b. The curved surface 342b is located on the front side 344b. Fixing elements 346b (two fixing elements 346b are shown in this embodiment) are formed on the back side 345b and are engageable with fixing elements 333b (two fixing elements 333b are shown in this embodiment) formed on the movable piece 33b. The fixing elements 346b are received in the fixing elements 333b to engage with the fixing elements 333b. In other embodiments, there can be any number of fixing elements 346b, such as one, two, three, four, and more, the number of the fixing element(s) 333b can be any that matches the number of the fixing elements 346b. The fixing element(s) 346b can be engaged with the fixing element(s) 333b in any suitable way. A fixing hole 347b is defined on the holding cover 34b. The fixing hole 347b is a through-hole and aligned with a fixing hole 327b defined on the seat 32b. A pin 35 can be inserted in the fixing holes 347b and 327b to fix the holding cover 34b to the seat 32b, thus fixing the movable piece 33b to the seat 32b after the catheter 5b is placed and held in the holding chamber 31b. When body fluid in a buffer zone 511b of the catheter 5b reaches a predefined maximum positon, the catheter holder 3b is rotated by the motor 2b to flow the body fluid in the catheter 5b to a body fluid collection bag connected to an end of the catheter 5b.

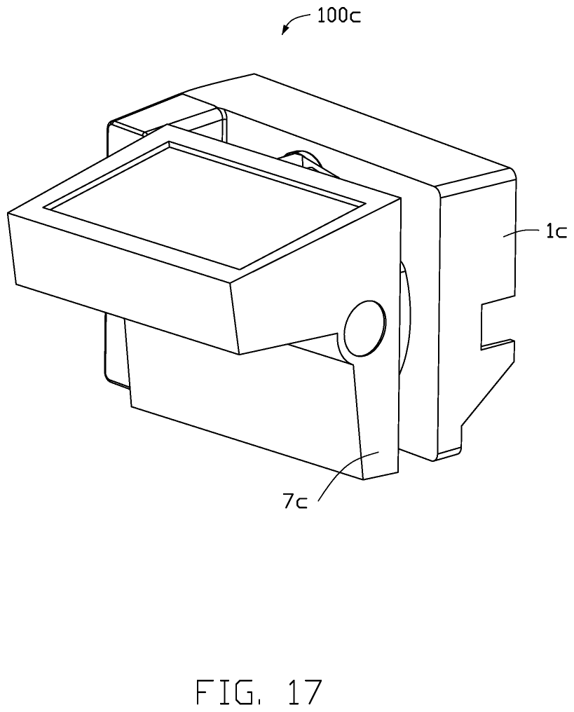

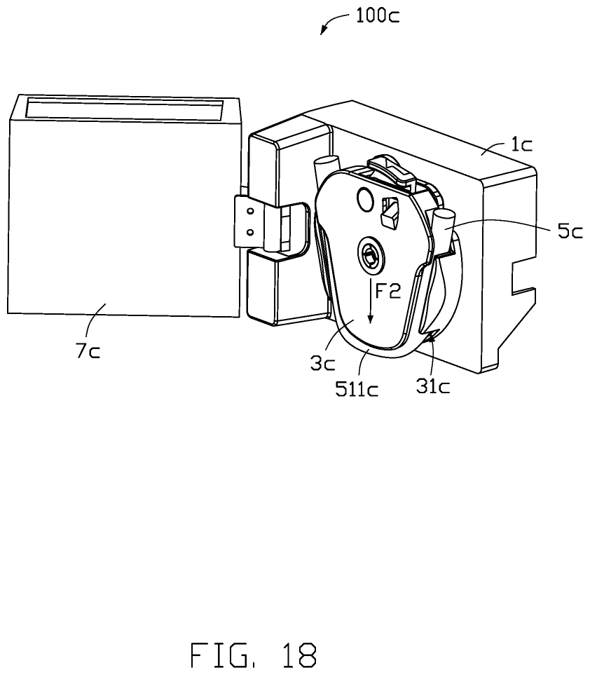

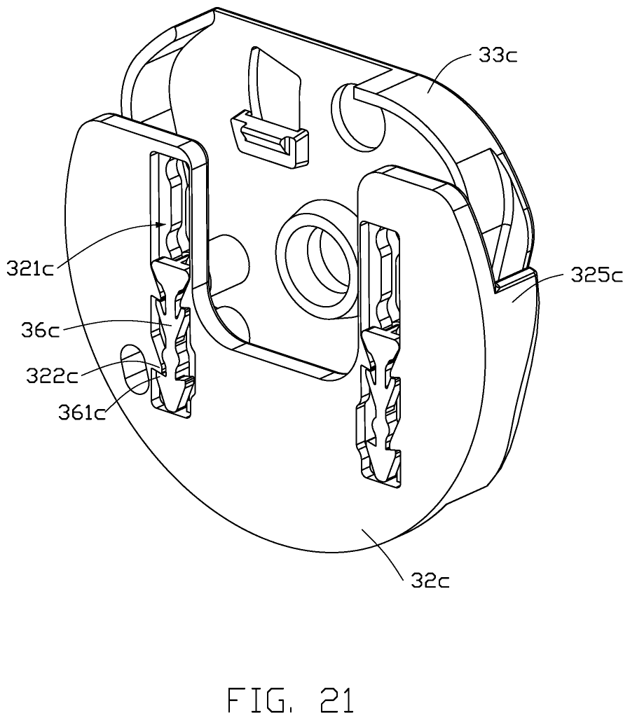

FIGS. 17-22 relates to a fourth embodiment. Referring to FIGS. 17-19, a device for monitoring body fluid discharge 100c (hereinafter “the device 100c”) includes a base 1c, a motor 2c, a catheter holder 3c, and a cover 7c. The motor 2c is fixed on the base 1c and includes an output shaft 21c. The catheter holder 3c is a rotatable disk located on the base 1c and rotatable by the output shaft 21c of the motor 2c. The cover 7c is pivotally connected to the base 1c and can be rotated to be open to expose the catheter holder 3c or be covering over the catheter holder 2c. When the cover 7c is opened, a catheter 5c can be mounted to a holding chamber 31c of the catheter holder 3c. When the cover 7c is closed, the cover 7c closes over the catheter 5c to seal the catheter 5c against spillage during use. When body fluid in a buffer zone 511c of the catheter 5c reaches a predefined maximum positon, the catheter holder 3c is rotated by the motor 2c to flow the body fluid in the catheter 5c to a body fluid collection bag connected to an end of the catheter 5c.

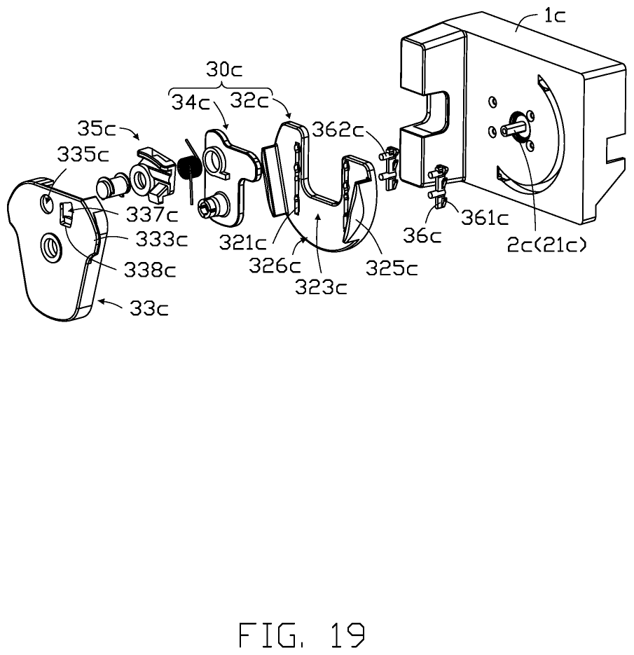

Referring to FIGS. 19-21, the catheter holder 3c includes a seat 30c, a movable piece 33c, and a pressing handle 35c. The seat 30c includes a first seat part 32c and a second seat part 34c. The second seat part 34c is located on the base 1c and rotatable by the output shaft 21c of the motor 2c. The first seat part 32c and the movable piece 33c can be assembled together, and the assembly of the first seat part 32c and the movable piece 33c can be assembled to the second seat part 34c by the pressing handle 35c. The assembly of the first seat part 32c and the movable piece 33c are rotatable by the second seat part 34c to empty out the body fluid in the catheter 5c. The assembly of the first seat part 32c and the movable piece 33c also forms a disposable part and can be detached from the second seat part 34c and disposed after use. The pressing handle 35c is assembled on the second seat part 34c and adapted to fix the first seat part 32c and the movable piece 33c to the second seat part 34c. The first seat part 32c includes at least one guiding groove 321c along which the movable piece 33c is movable. In the present embodiment, the first seat part 32c includes two guiding grooves 321c which are parallel and defined on a central portion 323c of the first seat part 32c. The first seat part 32c further includes at least one protruded block 325c (two protruded blocks 325c are shown in this embodiment) located at a surrounding portion 326c of the first seat part 32c. The protruded blocks 325c are symmetrically distributed on two sides of the guiding grooves 321c.

The catheter holder 3c further includes at least one guiding block 36c. The at least one guiding block 36c is engageable with the movable piece 33c and is receivable and movable in the at least one guiding groove 321c of the first seat part 32c, such that the movable piece 33c can be moved by users along the guiding groove 321c backwards. In the present embodiment, the catheter holder 3c includes two guiding blocks 36c corresponding to the two guiding grooves 321c. When the movable piece 33c engages with the guiding blocks 36c, and the guiding blocks 36c are received in the guiding grooves 321c of the first seat part 32c, the movable piece 33b is located between the protruded blocks 325c. The curved holding chamber 31c for holding a catheter 5c is formed between the movable piece 33c and the protruded blocks 325c. The movable piece 33c can be moved backward following the direction of arrow F2 to decrease a volume of the holding chamber 31c, so that the catheter 5c can be tightly held in the holding chamber 31c. In the present embodiment, each of the guiding grooves 321c is formed with at least one latching tooth 322c. The latching tooth 322c is protruded from a side of the guiding groove 321c and toward a central of the guiding groove 321c. Each of the guiding blocks 36c is formed with at least one matching tooth 361c corresponding to the at least one latching tooth 322c. The matching tooth 361c extends obliguely forwards from a side of the guiding blocks 36c. The guiding blocks 36c can be moved backwards by uses along the guiding grooves 321c, however, the guiding blocks 36c are prevented from moving forwards when the matching teeth 361c have moved to engage with the latching teeth 322c. Each of the guiding blocks 36c includes at least one leg 362c extending toward the movable piece 33c. The movable piece 33c is formed with at least one holes 331c for receiving the at least one leg 362c. When to assemble the movable piece 33c to the first seat part 32c, the guiding blocks 36c are placed and received in front sections of the guiding grooves 321c, thus to engaged with the guiding grooves 321c. The legs 362c of the guiding blocks 36c passes through the guiding grooves 321c. Free ends of the legs 362c are received in the holes 331c of the movable piece 33c and engaged with the movable piece 33c, thus to assemble the movable piece 33c and the first seat part 32c together. After the movable piece 33c and the first seat part 32c are assembled together, the curved holding chamber 31c is formed. The catheter 5c can be placed in the holding chamber 31c and the movable piece 33c can be moved backward following the direction of arrow F2. The guiding blocks 36c are moved by the movable piece 33c to rear sections of the guiding grooves 321c. The matching teeth 361c engage with the latching teeth 322c after the guiding blocks 36c reach the rear sections of the guiding grooves 321c, to prevent the guiding blocks 36c from being moved forward, thus to tightly held the catheter 5c in the holding chamber 31c.

The movable piece 33c further includes two flanks 333c located on opposite sides of the movable piece 33c. The flanks 333c cover the catheter 5c when the catheter 5c is received in the holding chamber 31c and prevent the catheter 5c from sliding out of the holding chamber 31c. The flanks 333c are symmetrical in this embodiment but may be asymmetrical in other embodiments.

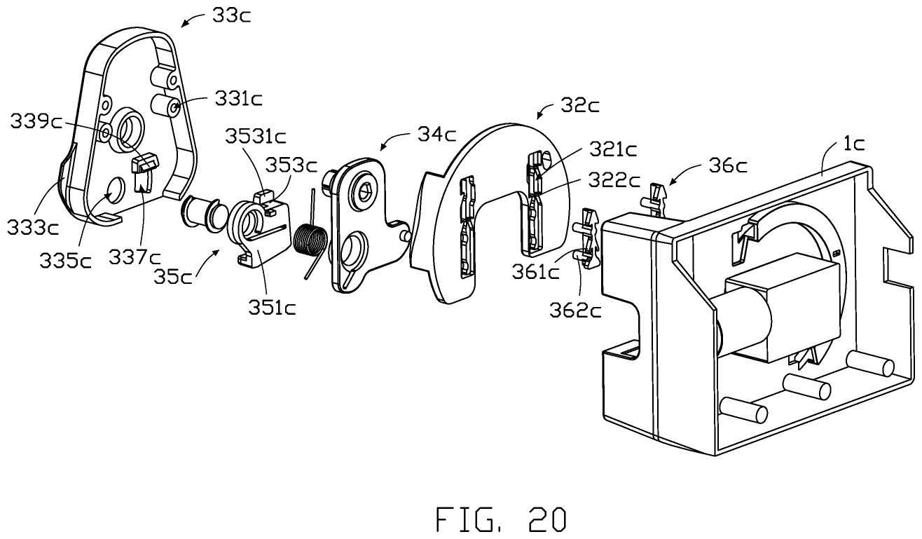

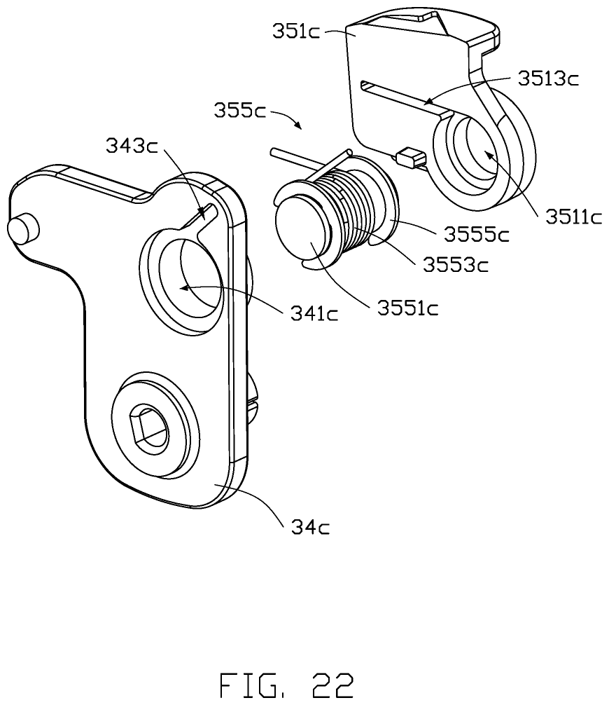

Referring to FIGS. 20 and 22, the pressing handle 35c includes a handle seat 351c, a handle 353c and a rotatable and restorable assembly 355c. The handle seat 351c is assembled on the second seat part 34c by the rotatable and restorable assembly 355c. The handle seat 351c can be rotated relative to the second seat part 34c and then restored because of the rotatable and restorable assembly 355c. The handle seat 351c defines a through hole 3511c which is aligned both with a through hole 341c defined on the second seat part 34c and a through hole 335c defined on the movable piece 33c. The through holes 341c, 3511c and 335c cooperatively receives the rotatable and restorable assembly 355c. The handle 353c is formed on the handle seat 351c and protrudes toward the movable piece 33c. A free end 3531c of the handle 353c is formed to be a hook that can hook the movable piece 33c, thus to fix the movable piece 33c and the first seat part 32c on the second seat part 34c. In the present embodiment, another through hole 337c is defined on the movable piece 33c. The free end 3531c of the handle 353c passes through the through hole 337c to hook an edge 338c of the through hole 337c. The movable piece 33c further includes a pressing tip 339c located besides the through hole 337c and protrudes towards the handle seat 351c. The pressing tip 339c is adapted to press the handle 353c to move (e.g., rotate) a little to pass the through hole 337c and hook the edge 338c of the through hole 337c.

The rotatable and restorable assembly 355c includes a shaft 3551c, a torsion spring 3553c sleeved on the shaft 3551c, and two limiting pieces 3555c to restrict a movement of the shaft 3551c along its axis direction. Two ends of the torsion spring 3553c are fixed by the handle seat 351c and the second seat part 34c respectively. In the present embodiment, grooves 3513c and 343c are formed on the handle seat 351c and the second seat part 34c for receiving and fixing the two ends of the torsion spring 3553c respectively. Two ends of the shaft 3551c extends out of the through holes 3511c and 341c respectively. The limiting pieces 3555c are fixed on the end of the shaft 3551c to restrict the movement of the shaft 3551c along its axis direction. In the present embodiment, the limiting pieces 3555c are C type retainer rings or E type retainer rings. When an external force is applied to the handle seat 351c or the handle 353c, for example, when the handle 353c is pressed by the pressing tip 339c, the handle seat 351c and the handle 353c can be rotated relative to the second seat part 34c by the external force. The rotation of the handle seat 351c and the handle 353c deforms the torsion spring 3553c. After the external force is removed, the torsion spring 3553c restores and rotates the handle seat 351c and the handle 353c back.

After the movable piece 33c and the first seat part 32c are assembled together, the assembly of the movable piece 33c and the first seat part 32c can be assembled to the second seat part 34c by the pressing handle 35c. During assembling, the assembly of the movable piece 33c and the first seat part 32c are aligned with the second seat part 34c and pressed toward the second seat part 34c by the users, the free end 3531c of the handle 353c is pressed by the pressing tip 339c. The handle 353c and the handle seat 351c rotate a little so that the handle 353c passes the through hole 337c. The rotation of the handle 353c and the handle seat 351c deforms the torsion spring 3553c. After the handle 353c passes through the through hole 337, the torsion spring 3553c restores and rotates the handle seat 351c and the handle 353c back, such that the free end 3531c of the handle 353c hooks the movable piece 33c. The assembly of the movable piece 33c and the first seat part 32c can also be dissembled from the second seat part 34c by applying an external force to the handle 353c. The handle 353c and the handle seat 351c rotate a little so that the handle 353c releases the movable piece 33c.

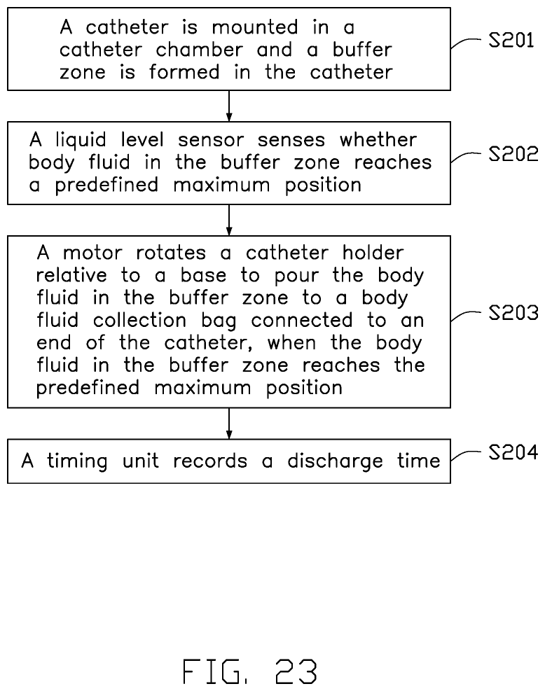

Referring to FIG. 23, a method for monitoring body fluid discharge is shown. The method may be carried out by any one of the devices 100, 100a, 100b, or 100c. The method may begin at block S201, in which a catheter is mounted in a catheter chamber (such as the catheter chamber 31, 31a, 31b or 31c) and a buffer zone (such as the buffer zone 511, 511a, 511b or 511c) is formed in the catheter. At block S202, a liquid level sensor (such as the liquid level sensor 4) senses whether body fluid in the buffer zone reaches a predefined maximum position. At block S203, a motor (such as the motor 2, 2b or 2c) rotates a catheter holder (such as the catheter holder 3 or 3a, 3b or 3c) relative to a base (such as the base 1, 1a, 1b or 1c) to pour the body fluid in the buffer zone to a body fluid collection bag connected to an end of the catheter, when the body fluid in the buffer zone reaches the predefined maximum position. At block S204, a timing unit records a discharge time.

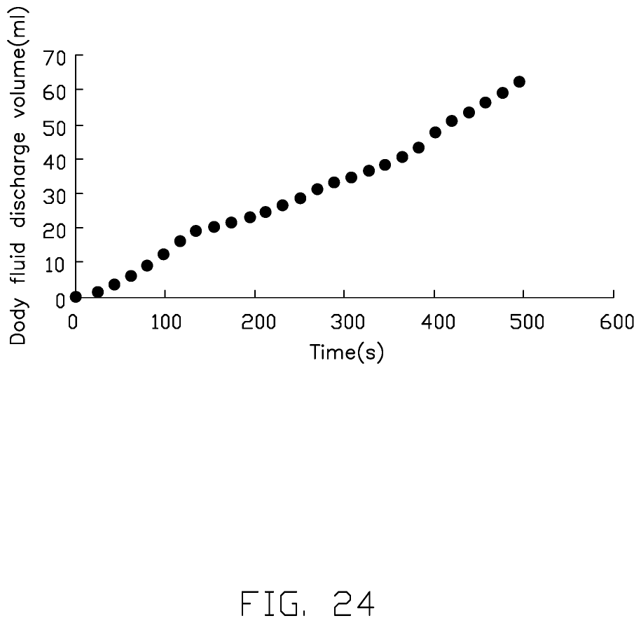

Referring to FIG. 24, a relationship between a volume of body fluid discharged to the buffer zone and time is shown. The volume of the body fluid discharged to the buffer zone increases as time moves on.

The method and device for monitoring body fluid discharge of the present disclosure employ a catheter holder to form a buffer zone on a catheter to store body fluid. When a liquid level sensor detects that the body fluid in the buffer zone reaches a predefined maximum position, the liquid level sensor triggers a motor to rotate the catheter holder to pour the body fluid to a body fluid collection bag connected to an end of the catheter, thus facilitating the monitoring of body fluid discharge.

The embodiments shown and described above are only examples. Even though numerous characteristics and advantages of the present technology have been set forth in the foregoing description, together with details of the structure and function of the present disclosure, the disclosure is illustrative only, and changes can be made in the detail, including in matters of shape, size, and arrangement of the parts within the principles of the present disclosure, up to and including the full extent established by the broad general meaning of the terms used in the claims.

What is claimed is:

1. A device for monitoring body fluid discharge comprising a base, a motor, a catheter holder and a liquid level sensor, the motor being fixed on the base and comprising an output shaft, the catheter holder being connected to the output shaft and rotatable relative to the base by the output shaft, the catheter holder comprising a curved holding chamber with two openings configured for receiving a catheter, and the liquid level sensor being disposed at one of the openings and adapted for detecting a liquid level of body fluid in the catheter.

2. The device as claimed in claim 1, wherein the catheter holder comprises linkage assembly and a pivot seat, the linkage assembly is configured to push or pull the pivot seat to rotate relative to the base, the holding chamber is formed on the pivot seat.

3. The device as claimed in claim 2, wherein the linkage assembly comprises a first linkage rod and a second linkage rod, an end of the first linkage rod is connected to the motor and another end of the first linkage rod is connected to an end of the second linkage rod, the pivot seat comprises a front side and a back side opposite to the front side, the holding chamber is formed at the front side, and another end of the second linkage rod is connected to the back side.

4. The device as claimed in claim 1, the catheter holder is a rotatable disk mounted on the base and comprises a disk and a plurality of spring clips mounted around the disk, the holding chamber is formed between the disk and the spring clips.

5. The device as claimed in claim 4, wherein a circumferential surface of the disk facing the spring clips defines grooves, each of the grooves corresponding to one of the spring clips.

6. The device as claimed in claim 5, wherein the catheter holder comprises two spring clips and the two spring clips are symmetrical about the disk.

7. The device as claimed in claim 6, wherein the base is formed with an upper hanging ring and a lower hanging ring.

8. The device as claimed in claim 7, wherein the device further comprises a cover pivotally connected to the base.

9. The device as claimed in any one of claims 1, wherein the holding chamber is a U-shape.

10. The device as claimed in claim 1, wherein the catheter holder comprises a seat rotatable by the output shaft and a movable piece movable on the seat, the seat comprises at least one guiding groove along which the movable piece moves, a volume of the holding chamber is changeable by moving the movable piece along the guiding groove.

11. The device as claimed in claim 10, wherein a quantity of the at least one guiding groove is two, the seat further comprises a platform protruding from a central portion of the seat and a pressing plate covering the platform, the pressing plate has two opposite edges exceeding beyond the platform to form the guiding grooves at opposite sides of the platform.

12. The device as claimed in claim 11, wherein the movable piece comprises two opposite guiding blocks engageable with the guiding grooves, the movable piece is U-shaped comprising two legs, the guiding blocks are formed on the legs.

13. The device as claimed in claim 10, wherein the seat further comprises at least one protruded block at a surrounding portion of the seat, the holding chamber is formed between the protruded block and the movable piece.

14. The device as claimed in claim 10, wherein the device further comprises a pin and a holding cover fixed on the movable piece and covering the movable piece, a fixing hole is defined on the holding cover, the fixing hole is a through-hole and aligned with a fixing holing defined on the seat, both of the fixing holes engage with the pin to fix the holding cover and the movable piece to the seat after the catheter is received in the holding chamber.

15. The device as claimed in claim 14, wherein one of the holding cover and the movable piece comprises two flanks on opposite sides thereof, and the flanks cover the catheter when the catheter is received in the holding chamber and prevent the catheter from sliding out of the holding chamber.

16. The device as claimed in claim 10, wherein the seat comprises a first seat part and a second seat part, the second seat part is located on the base and rotatable by the output shaft, the first seat part is engaged with the second seat part and is rotatable by the second seat part, the movable piece is movable on the first seat part, the first seat part and the moveable piece are detachable from the second seat part and disposable.

17. The device as claimed in claim 16, wherein the catheter holder further comprises a pressing handle assembled on the second seat part to fix the first seat part and the movable piece to the second seat part, the pressing handle comprising a handle seat, a handle and a rotatable and restorable assembly, the handle seat is assembled on the second seat part by the rotatable and restorable assembly, the handle is formed on the handle seat and protrudes toward the movable piece, the rotatable and restorable assembly allows the handle and the handle seat to rotate relative to the second seat part and then restore, thus enables the handle to hook the movable piece or release the movable piece.

18. The device as claimed in claim 10, wherein the catheter holder comprises at least one guiding block, the guiding block is engageable with the movable piece and is receivable and movable in the guiding groove, the guiding block is formed with at least one matching tooth corresponding to at least one latching tooth extended from a side of the guiding groove, the guiding block is stopped to move when the matching tooth engages with the latching tooth.

19. The device as claimed in claim 1, wherein when the liquid level sensor detects that the body fluid in the catheter reaches a predefined maximum position, the liquid level sensor sends a signal to trigger the motor to rotate the catheter holder to pour the body fluid in the catheter.

20. A method for monitoring body fluid discharge using the device as claimed in claim 1, the method comprising:
mounting a catheter in the catheter chamber and forming a buffer zone in the catheter;
sensing whether the body fluid in the buffer zone reaches a predefined maximum position; and
rotating the catheter holder relative to the base to pour the body fluid in the buffer zone to a body fluid collection bag connected to an end of the catheter, when the body fluid in the buffer zone reaches the predefined maximum position.

21. The method as claimed in claim 20, further comprising recording a current time point as a discharge time when the body fluid in the buffer zone reaches the predefined maximum position.
